(19) United States
(12) Patent Application Publication
(10) Pub. No.: US 2026/0041432 A1
(43) Pub. Date: Feb. 12, 2026

(54) SELF SEALING PROXIMAL MICROCATHETER AND ACCESS AID

(71) Applicant: Neuravi Limited, Galway (IE)

(72) Inventors: Karl KEATING, Galway (IE); David VALE, Barna (IE); Brendan CASEY, Galway (IE); Ronald KELLY, Galway (IE)

(73) Assignee: Neuravi Limited, Galway (IE)

(21) Appl. No.: 18/800,386

(22) Filed: Aug. 12, 2024

(51) Int. Cl. A61B 17/12; A61M 25/01; A61M 25/09

(52) U.S. Cl. CPC A61B 17/12136; A61B 17/1204; A61B 17/12109; A61M 25/0108; A61M 25/09; A61B 2017/1205

(57) ABSTRACT

A retrograde blood flow occlusion flushing device includes a first catheter, and a second catheter. The second catheter is axially movable with respect to the first catheter. The second catheter has an inflatable balloon connected to an outer circumferential surface of the second catheter at the distal end of the second catheter. The balloon is movable between a collapsed state and an inflated state. The second catheter has a port in the sidewall between the proximal end of the balloon and the distal end of the balloon. The balloon has at least one port proximate the proximal end of the balloon. In the inflated state, fluid is deliverable through the lumen of the second catheter, through the port in the sidewall of the second catheter to inflate the balloon and at least some of the fluid exits the inflated balloon in the proximal direction through the port in the balloon.

### FIELD

The present disclosure generally relates to medical instruments, and more particularly, to a catheter with enhanced navigability and retrograde flushing.

### BACKGROUND

Various types of catheters have been developed for use during neurovascular interventions. No matter which specific type of catheter is selected, a catheter needs to be able to track through a specific anatomy to reach a treatment site. Then depending on the functionality of the selected catheter, an implant, another catheter, or other device may be put into and movable inside along its lumen to implement the designated task. For example, an aspiration catheter is known as an intermediate or distal access catheter. Such aspiration catheter can be used in mechanical thrombectomy procedures to perform, for example, a direct contact aspiration. In this case, an aspiration catheter navigates through the vascular anatomy to reach the occlusion site, with its proximal end connected to a syringe or aspiration pump which generates negative pressure to engage or ingest the thrombus into the aspiration catheter.

During the process of navigating through the vasculature of a patient, factors such as efficiency and navigability are main factors to evaluate a performance of a catheter. Navigability is a desired character especially for distal access catheters (e.g., to ease of navigation and effective aspiration). However, this can be challenging due to the nature of the vasculature in which the catheter is navigating. Therefore, there is a need for improved or enhanced navigability for various catheters.

During the process, once the aspiration catheter has traveled as far into the vasculature as reasonably possible, the thrombus may be further distal within the vasculature from the distal end of the aspiration catheter. In such a scenario, there is a need to help keep the vessel from collapsing due to the aspiration and to also dislodge the thrombus from the vasculature.

### SUMMARY

Disclosed herein are various exemplary catheters with enhanced navigability and flushing capabilities, which may at least alleviate above needs. Also, a corresponding method for steering a catheter and retrograde flushing of a vessel is provided in this disclosure.

In accordance with an aspect of the present disclosure a retrograde blood flow occlusion flushing device includes a first catheter, and a second catheter. The first catheter has a proximal end, a distal end, and a lumen extending through the first catheter from the proximal end to the distal end. The second catheter is disposed within the lumen of the first catheter and has a proximal end, a distal end, and a lumen extending through the second catheter from the proximal end to the distal end. The second catheter is axially movable with respect to the first catheter.

The second catheter has an inflatable balloon connected to an outer circumferential surface of the second catheter at the distal end of the second catheter. The balloon is movable between a collapsed state and an inflated state. The balloon is fixedly connected to the outer circumferential surface of second catheter at a proximal end of balloon and at a distal end of the balloon. The second catheter has a port in the sidewall between the proximal end of the balloon and the distal end of the balloon. The balloon has at least one port proximate the proximal end of the balloon. The balloon is movable between a collapsed state and an inflated state. The balloon can be inflated by a fluid deliverable through the lumen of the second catheter via the proximal end and exiting the port in the sidewall of the second catheter to selectively move the balloon between the collapsed state and the inflated state. In the inflated state, the fluid is deliverable through the lumen of the second catheter, through the port in the sidewall of the second catheter to inflate the balloon and at least some of the fluid exits the inflated balloon in the proximal direction through the at least one port in the balloon.

In accordance with another aspect of the present disclosure, a blood flow occlusion flushing device includes a first catheter having a proximal end, a distal end, and a lumen extending through the first catheter from the proximal end to the distal end. A second catheter is disposed within the lumen of the first catheter. The second catheter has a proximal end, a distal end, and a lumen extending through the second catheter from the proximal end to the distal end. The second catheter is axially movable with respect to the first catheter. The second catheter has a port in the sidewall proximate the distal end of the second catheter. A guidewire is disposed within the lumen of the second catheter. The guidewire is axially movable with respect to the second catheter. The guidewire has a proximal end, and a distal end. The guidewire has a first diameter that is less than an inner diameter of the second catheter. The guidewire has a radially outwardly projecting bump proximate the distal end of guidewire. The radially outwardly projecting bump has an outer diameter that is approximately equal to an inner diameter of the second catheter at the distal end of the second catheter. When the radially outwardly projecting bump is aligned with the distal end of the second catheter, the radially outwardly projecting bump is in sealing contact with the inner diameter of the second catheter at the distal end of the second catheter. In this position, a fluid can be delivered through the lumen of the second catheter via the proximal end and exit the port in the sidewall of the second catheter.

In accordance with another aspect of the present disclosure, a blood flow occlusion flushing device includes a first catheter, a second catheter, and a guidewire. The first catheter has a proximal end, a distal end, and a lumen extending through the first catheter from the proximal end to the distal end. The second catheter is disposed within the lumen of the first catheter. The second catheter has a proximal end, a distal end, and a first lumen extending through the second catheter from the proximal end to the distal end. A second lumen extends through the second catheter from the proximal end to the distal end. The second catheter is axially movable with respect to the first catheter. The second catheter has a port in the sidewall proximate the distal end of the second catheter. The second catheter has an inflatable balloon connected to an outer circumferential surface of the second catheter at the distal end of the second catheter. The balloon is movable between a collapsed state and an inflated state. The balloon is fixedly connected to the outer circumferential surface of the second catheter at a proximal end of the balloon and at a distal end of the balloon. The second catheter has at least one port in the sidewall between the proximal end of the balloon and the distal end of the balloon. The balloon has at least one port proximate to the proximal end of the balloon. A guidewire is disposed within the second lumen of the second catheter. The guidewire is axially movable with respect to the second catheter. The guidewire has a proximal end and a distal end. The balloon is movable between the collapsed state and the inflated state by a fluid that can be delivered through the lumen of the second catheter via the proximal end and exiting the port in the sidewall of the second catheter to selectively move the balloon between the collapsed state and the inflated state. In the inflated state, fluid is deliverable through the first lumen of the second catheter, through the port in the sidewall of the second catheter to inflate the balloon and at least some of the fluid exits the inflated balloon in the proximal direction through the at least one port in the balloon.

### BRIEF DESCRIPTION OF THE DRAWINGS

The above and further aspects of this disclosure are further discussed with reference to the following description in conjunction with the accompanying drawings, in which like numerals indicate like structural elements and features in various figures. The drawings are not necessarily to scale, emphasis instead being placed upon illustrating principles of the disclosure. The figures depict one or more implementations of the inventive devices, by way of example only, not by way of limitation.

FIG. 1A is a cross-sectional view of vessels within the vasculature and having a retrograde blood flow occlusion flushing device illustrated within a vessel for removing an obstruction in the vessel according to aspects of the present disclosure.

FIG. 1B is an enlarged view of circle 1B from FIG. 1A according to aspects of the present disclosure.

FIG. 2 illustrates the flushing device navigating by branch vessels within the vasculature according to aspects of the present disclosure.

FIG. 3 is a plan view of the distal section of the flushing device, with parts broken away, according to aspects of the present disclosure.

FIG. 4 is a cross-sectional view taken along lines 4-4 of FIG. 3 and looking in the direction of the arrows according to aspects of the present disclosure.

FIG. 5 is a cross-sectional view of a distal section of the flushing device taken along lines 5-5 of FIG. 4 and looking in the direction of the arrows according to aspects of the present disclosure.

FIG. 6 is a cross-sectional view of a distal section of the flushing device showing a balloon in an inflated state according to aspects of the present disclosure.

FIG. 7 is a cross-sectional view taken along lines 7-7 of FIG. 6 and looking in the direction of the arrows according to aspects of the present disclosure.

FIG. 8A is a cross-sectional view of a distal section of another embodiment of the flushing device according to aspects of the present disclosure.

FIG. 8B is a cross-sectional view of a distal section of the flushing device shown in FIG. 8A illustrating a balloon in an inflated state according to aspects of the present disclosure.

FIG. 9A is a cross-sectional view of a distal section of another embodiment of the flushing device according to aspects of the present disclosure.

FIG. 9B is a cross-sectional view of a distal section of the flushing device shown in FIG. 9A according to aspects of the present disclosure.

FIG. 10A is a cross-sectional view of a distal section of another embodiment of the flushing device according to aspects of the present disclosure.

FIG. 10B is a cross-sectional view of a distal section of the flushing device shown in FIG. 10A illustrating a balloon in an inflated state according to aspects of the present disclosure.

FIG. 11A is a cross-sectional view of a distal section of another embodiment of the flushing device according to aspects of the present disclosure.

FIG. 11B is a cross-sectional view of a distal section of the flushing device shown in FIG. 10A illustrating a balloon in an inflated state according to aspects of the present disclosure.

FIG. 12 is a flow chart illustrating a method of removing an obstruction from a vessel.

FIG. 13 is a continuation of the flow chart shown in FIG. 12 illustrating a method of removing an obstruction from a vessel.

FIG. 14 is a continuation of the flow chart shown in FIG. 13 illustrating a method of removing an obstruction from a vessel.

FIG. 15 is a continuation of the flow chart shown in FIG. 14 illustrating a method of removing an obstruction from a vessel.

### DETAILED DESCRIPTION

As used herein, the terms “about” or “approximately” for any numerical values or ranges indicate a suitable dimensional tolerance that allows the part or collection of components to function for its intended purpose as described herein. More specifically, “about” or “approximately” may refer to the range of values ±20% of the recited value, e.g. “about 80%” may refer to the range of values from 61% to 99%.

As used herein, the term “microcatheter” is a catheter having a diameter that is small in comparison to catheters in cardiovascular applications, i.e., 8 French or less.

As used herein, the terms “tubular” and “tube” are to be construed broadly and are not limited to a structure that is a right cylinder or strictly circumferential in cross-section or of a uniform cross-section throughout its length. For example, a tubular structure or system is generally illustrated as a substantially right cylindrical structure. However, the tubular system may have a tapered or curved outer surface without departing from the scope of the present disclosure.

Documents incorporated by reference in the present patent application are to be considered an integral part of the application except that to the extent any terms are defined in these incorporated documents in a manner that conflicts with the definitions made explicitly or implicitly in the present specification, only the definitions in the present specification should be considered.

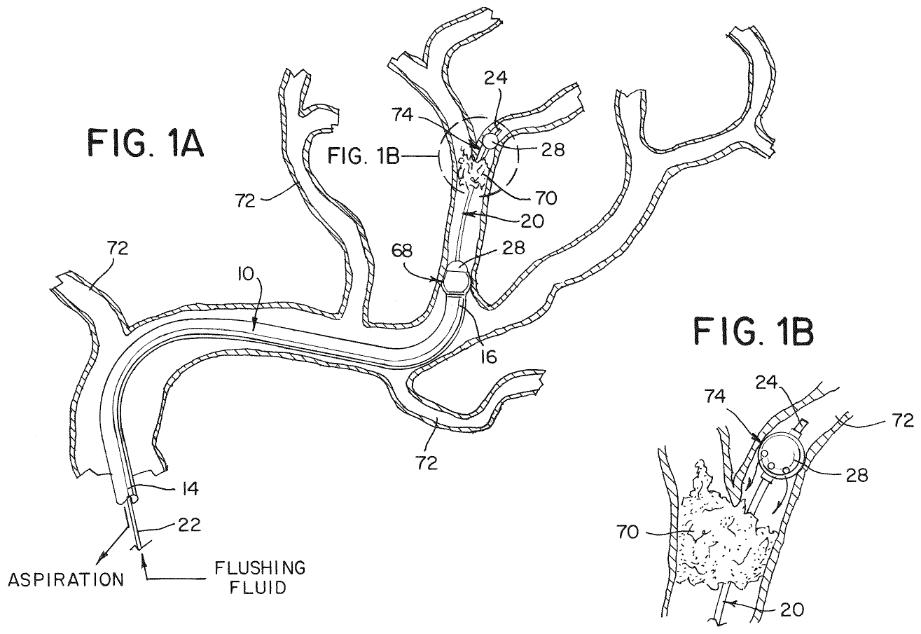

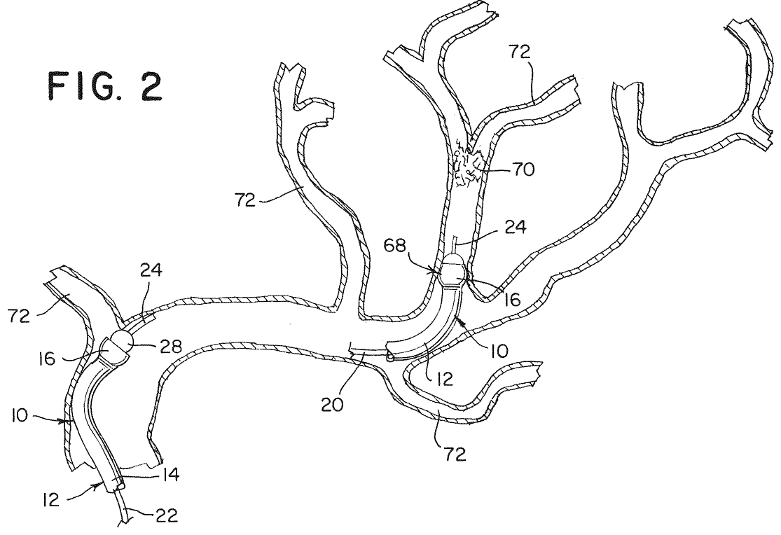

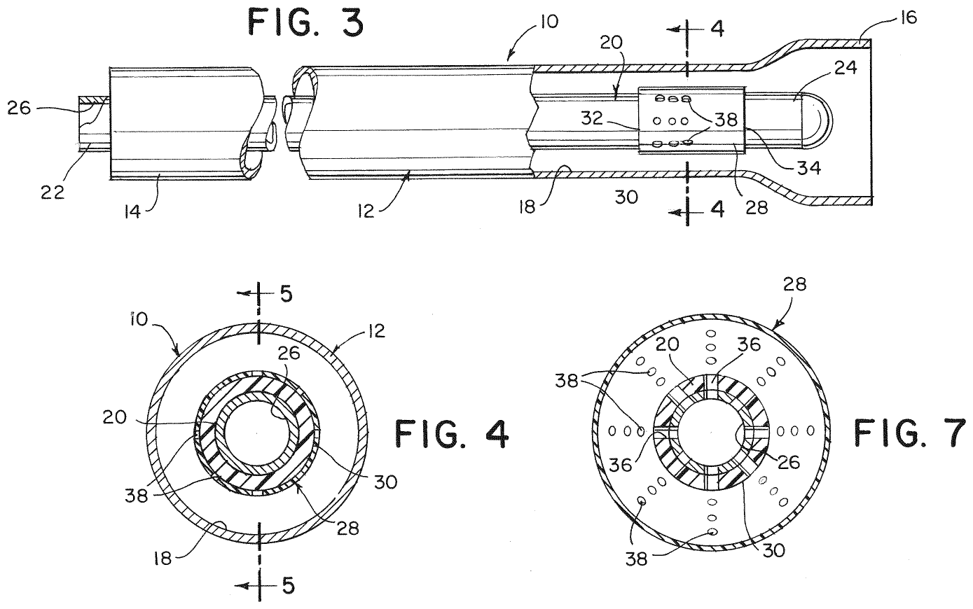

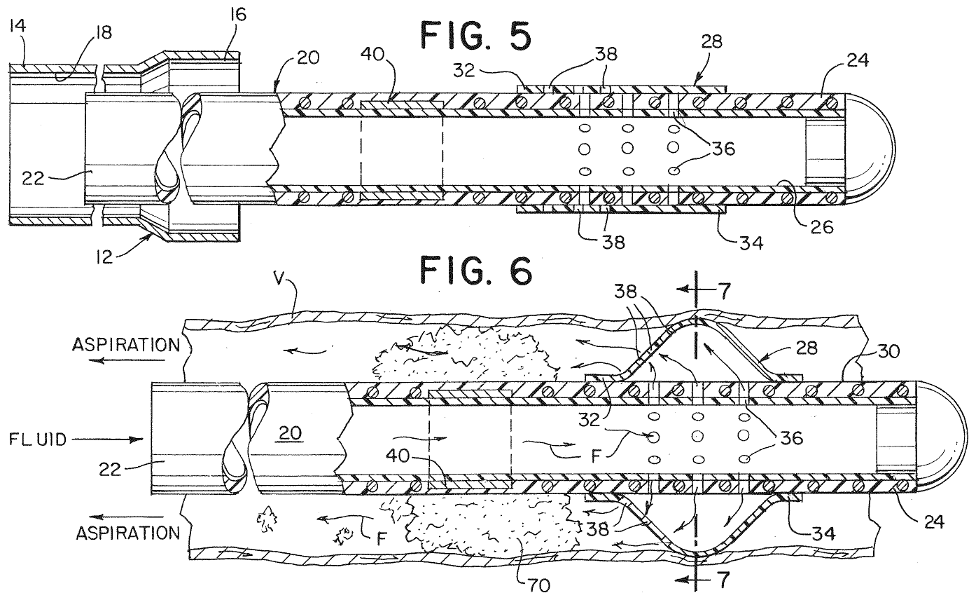

Referring now to FIGS. 1A-7, a retrograde blood flow occlusion flushing device 10 is illustrated. Device 10 includes a first catheter 12, and a second catheter 20. First catheter 12 has a proximal end 14, a distal end 16, and a lumen 18 extending through the first catheter 12 from the proximal end 14 to the distal end 16. Second catheter 20 is disposed within the lumen 18 of the first catheter 12. Second catheter 20 has a proximal end 22, a distal end 24, and a lumen 26 extending through the second catheter 20 from the proximal end 22 to the distal end 24. Second catheter 20 is axially movable with respect to the first catheter 12. Second catheter 20 has an inflatable balloon 28 connected to an outer circumferential surface 30 of the second catheter 20 at the distal end 24 of the second catheter 20. Balloon 28 is movable between a collapsed state and an inflated state. Balloon 28 is fixedly connected to the outer circumferential surface 30 of second catheter 20 at a proximal end 32 of balloon 28 and at a distal end 34 of the balloon. Second catheter 20 has at least one port 36 in the sidewall between the proximal end 32 of the balloon 28 and the distal end 34 of the balloon 28. Balloon 28 has at least one port 38 proximate the proximal end 32 of the balloon.

Balloon 28 is movable between a collapsed state and an inflated state. Balloon 28 can be inflated by a fluid F deliverable through the lumen 26 of the second catheter 20 via the proximal end 22 and exiting the port 36 in the sidewall of the second catheter 20 to selectively move the balloon 28 between the collapsed state and the inflated state. In the inflated state, the fluid F is deliverable through the lumen 26 of the second catheter, through the port 36 in the sidewall of the second catheter 20 to inflate the balloon 28 and at least some of the fluid F exits the inflated balloon 28 in the proximal direction through the at least one port 38 in the balloon 28.

The distal end 24 of the second catheter 20 is sealed such that the only openings in the second catheter are at the proximal end 22 and at the port 36 in the sidewall of the second catheter 20. The second catheter 20 has a first radiopaque marker 40 proximate to and proximal to the balloon 28. The second catheter 20 has a second radiopaque marker 42 proximate to and distal to the balloon 28. The second radiopaque marker 42 is disposed at the sealed distal end of the second catheter 20. The distal end 16 of the first catheter 12 has a radially outwardly flared opening 44.

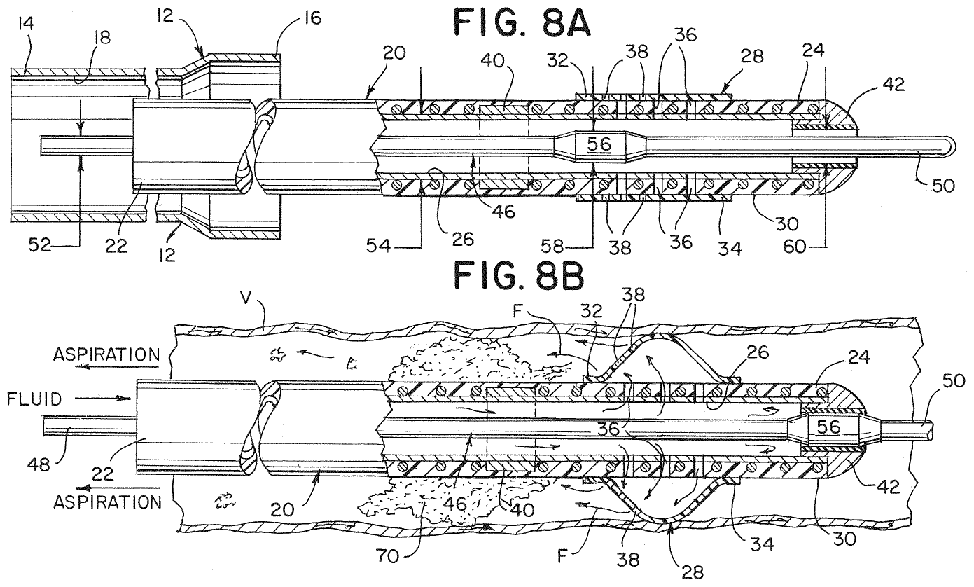

Referring now to FIGS. 8A and 8B, a blood flow occlusion flushing device 10′is illustrated. Elements that are the same as the embodiment from FIGS. 1-7, will not be described in further detail for the sake of brevity in the disclosure. Like the embodiment shown in FIGS. 1A-7, device 10′includes a first catheter 12 having a proximal end 14, a distal end 16, and a lumen 18 extending through the first catheter 12 from the proximal end 14 to the distal end 16. A second catheter 20 is disposed within the lumen 18 of the first catheter 12. Second catheter 20 has a proximal end 22, a distal end 24, and a lumen 26 extending through the second catheter 20 from the proximal end 22 to the distal end 24. Second catheter 20 is axially movable with respect to the first catheter 12. Second catheter 20 has a port 36 in the sidewall proximate the distal end 24 of the second catheter 20.

A guidewire 46 is disposed within the lumen 18 of the second catheter 20. Guidewire 46 is axially movable with respect to the second catheter 20. Guidewire 46 has a proximal end 48, and a distal end 50. Guidewire 46 has a first diameter 52 that is less than an inner diameter 54 of the second catheter 20. Guidewire 46 has a radially outwardly projecting bump 56 proximate the distal end 50 of guidewire 46. The radially outwardly projecting bump 56 has an outer diameter 58 that is approximately equal to an inner diameter 60 of the second catheter 20 at the distal end 24 of the second catheter 20. When the radially outwardly projecting bump 56 is axially aligned with the distal end 24 of the second catheter 20 as shown, for example, in FIG. 8B, the radially outwardly projecting bump 56 is in sealing contact with the inner diameter 60 of the second catheter 20 at the distal end 24 of the second catheter 20. In this position, a fluid F can be delivered through the lumen 18 of the second catheter 20 via the proximal end 22 and exit the port 36 in the sidewall of the second catheter 20. Fluid F can be, for example, saline. The radially outwardly projecting bump 56 can also be a radiopaque marker.

The blood flow occlusion flushing device 10 can include an inflatable balloon 28 connected to an outer circumferential surface 30 of the second catheter 20 at the distal end 24 of the second catheter 20. Balloon 28 is movable between a collapsed state as shown in FIG. 8A, and an inflated state, as shown in FIG. 8B. Inflatable balloon 28 is fixedly connected to the outer circumferential surface 30 of second catheter 20 at a proximal end 32 of balloon 28 and at a distal end 34 of balloon 28. Balloon 28 has at least one port 38 between the proximal end 32 of the balloon and the distal end 34 of the balloon 28. The at least one port 38 in the balloon being disposed at the proximal end 32 of the balloon 28. Second catheter 20 has a first radiopaque marker 40 proximate to and proximal to the balloon 28. Second catheter 28 also can have a second radiopaque marker 42 proximate to and distal to the balloon 28.

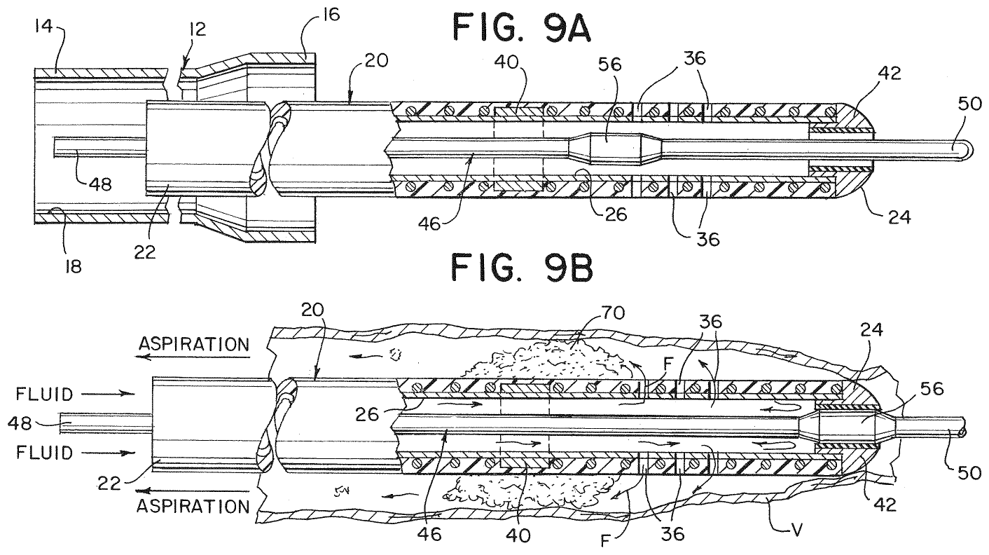

Referring now to FIGS. 9A and 9B, a blood flow occlusion flushing device 10″ is illustrated. Elements that are the same as the embodiment from FIGS. 1-7, and FIGS. 8A-8B, will not be described in further detail for the sake of brevity in the disclosure. Like the embodiment shown in FIGS. 1A-7, and FIGS. 8A-8B, device 10″ includes a first catheter 12, and a second catheter 20. Device 10″ also includes a guidewire 46. First catheter 12 has a proximal end 14, a distal end 16, and a lumen 18 extending through the first catheter 12 from the proximal end 14 to the distal end 16. Second catheter 20 is disposed within the lumen 18 of the first catheter 12. Second catheter 20 has a proximal end 22, a distal end 24, a first lumen 26 extending through the second catheter 20 from the proximal end 22 to the distal end 24. Second catheter 20 is axially movable with respect to the first catheter 12. Second catheter 20 has at least one port 36 in the sidewall proximate the distal end 34 of the second catheter 20.

A guidewire 46 is disposed within the lumen 26 of the second catheter 20. Guidewire 46 is axially movable with respect to the second catheter 20. Guidewire 46 has a proximal end 48, and a distal end 50. Guidewire 46 has a first diameter 52 that is less than an inner diameter 54 of the second catheter 20. Guidewire 46 has a radially outwardly projecting bump 56 proximate the distal end 50 of guidewire 46. The radially outwardly projecting bump 56 can also be a radiopaque marker. The radially outwardly projecting bump 56 has an outer diameter 58 that is approximately equal to an inner diameter 60 of the second catheter 20 at the distal end 24 of the second catheter 20. When the radially outwardly projecting bump 56 is axially aligned with the distal end 24 of the second catheter 20 as shown, for example, in FIG. 9B, the radially outwardly projecting bump 56 is in sealing contact with the inner diameter 60 of the second catheter 20 at the distal end 24 of the second catheter 20. In this position, a fluid F can be delivered through the lumen 26 of the second catheter 20 via the proximal end 22 and exit the port 36 in the sidewall of the second catheter 20. In this example, the vessel V may not form a seal with the distal end of the second catheter, but fluid F may still dislodge a thrombus or clot 70. In some examples, vessel V may seal about the distal end of second catheter 20 and fluid F may move clot 70 proximally with the aspiration force being applied to help remove clot 70 into the distal end of the aspiration catheter 12 so that the clot can be removed from the vasculature.

To facilitate the seal between the bump 56 and inner diameter 60 of the second catheter as shown in FIG. 9B, the bump may be made from an elastomeric material or may have a hydrophilic coating on it. Likewise with the tip of the microcatheter. Another option is that you don't need an absolute seal, there could be a small amount of fluid that travels distally but when balanced with the compliance of the balloon and the pore size on the proximal side of the balloon, most of the fluid travels proximally to push the clot to the funnel like opening at the distal end of the first catheter.

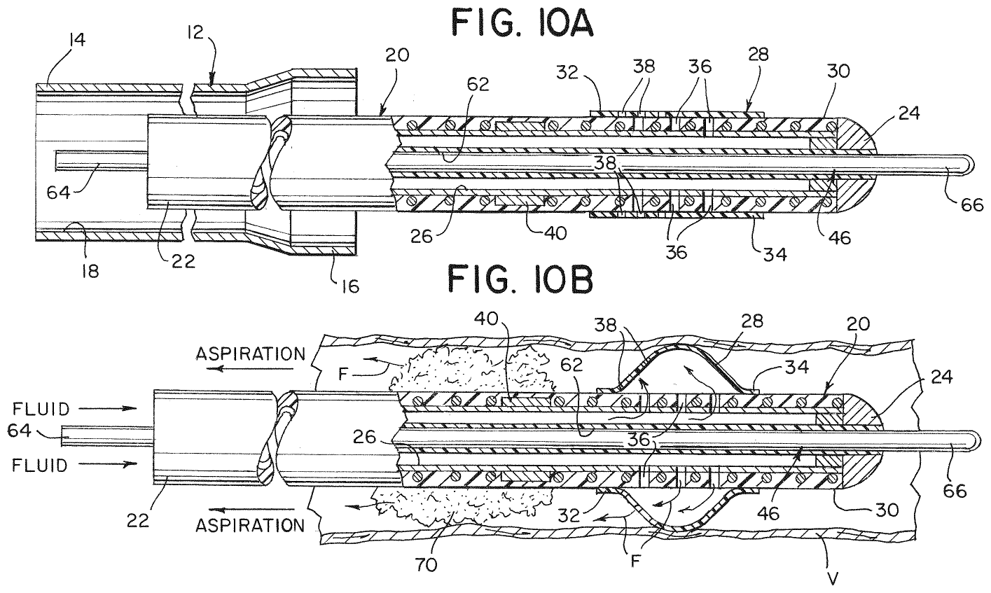

Referring now to FIGS. 10A and 10B, a blood flow occlusion flushing device 10″′ is illustrated. Elements that are the same as the embodiment from FIGS. 1-7, FIGS. 8A-8B, and FIGS. 9A-9B will not be described in further detail for the sake of brevity in the disclosure. Like the embodiment shown in FIGS. 1A-7, FIGS. 8A-8B, and FIGS. 9A-9B device 10″′ includes a first catheter 12, and a second catheter 20. Device 10″′ also includes a guidewire 46. First catheter 12 has a proximal end 14, a distal end 16, and a lumen 18 extending through the first catheter 12 from the proximal end 14 to the distal end 16. Second catheter 20 is disposed within the lumen 18 of the first catheter 12. Second catheter 20 has a proximal end 22, a distal end 24, a first lumen 26 extending through the second catheter 20 from the proximal end 22 to the distal end 24. A second lumen 62 extends through the second catheter 20 from the proximal end 22 to the distal end 24. A guidewire 46 is axially slidably received within second lumen 62. First lumen 26 is disposed coaxially about guidewire 46. Guidewire 46 has a proximal end 48, and a distal end 50. Second catheter 20 is axially movable with respect to the first catheter 12. Second catheter 20 has at least one port 36 in the sidewall proximate the distal end 34 of the second catheter 20. Second catheter 20 has an inflatable balloon 28 connected to an outer circumferential surface 30 of the second catheter 20 at the distal end 24 of the second catheter 20. Balloon 28 is movable between a collapsed state and an inflated state. Balloon 28 is fixedly connected to the outer circumferential surface 30 of the second catheter 20 at a proximal end 32 of the balloon 28 and at a distal end 34 of the balloon 28. Second catheter 20 has the at least one port 36 in the sidewall disposed between the proximal end 32 of the balloon 28 and the distal end 34 of the balloon 28. Balloon 28 has at least one port 38 proximate to the proximal end 32 of the balloon 28 as shown in FIG. 10B.

Balloon 28 is movable between the collapsed state and the inflated state by a fluid F that can be delivered through the lumen 26 of the second catheter 20 via the proximal end 22 and exiting the port 36 in the sidewall of the second catheter 20 to selectively move the balloon 28 between the collapsed state and the inflated state. In the inflated state, fluid F is deliverable through the first lumen 26 of the second catheter, through the port 36 in the sidewall of the second catheter 20 to inflate the balloon 28 and at least some of the fluid F exits the inflated balloon 28 in the proximal direction through the at least one port 38 in the balloon 28 as shown in FIG. 10B.

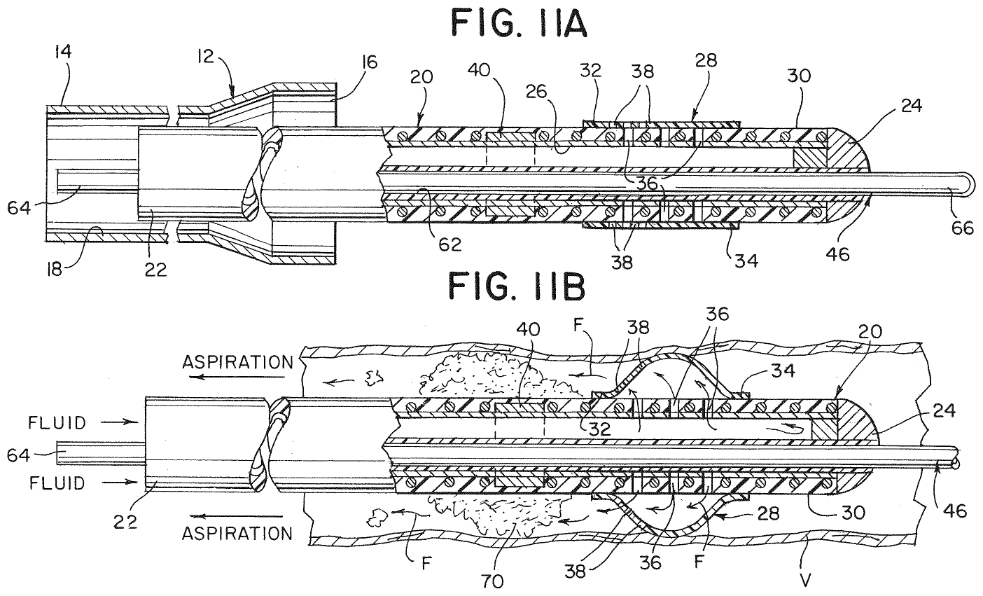

The first lumen 26 and the second lumen 62 in the second catheter 20 can be co-axial. Alternatively, the second lumen 62 in the second catheter 20 can be offset with respect to the first lumen 26 in the second catheter 20 as illustrated in FIGS. 11A and 11B. The distal end 24 of the second catheter 20 is sealed such that the only openings in the second catheter 20 are at the proximal end 22 and at the port 36 in the sidewall of the second catheter 20.

Second catheter 20 has a first radiopaque marker 40 proximate to and proximal to the balloon 28. Second catheter 20 has a second radiopaque marker 42 proximate to and distal to the balloon 28. Second radiopaque marker 42 is disposed at the sealed distal end 24 of the balloon. The distal end 16 of the first catheter has a radially outwardly flared opening 44.

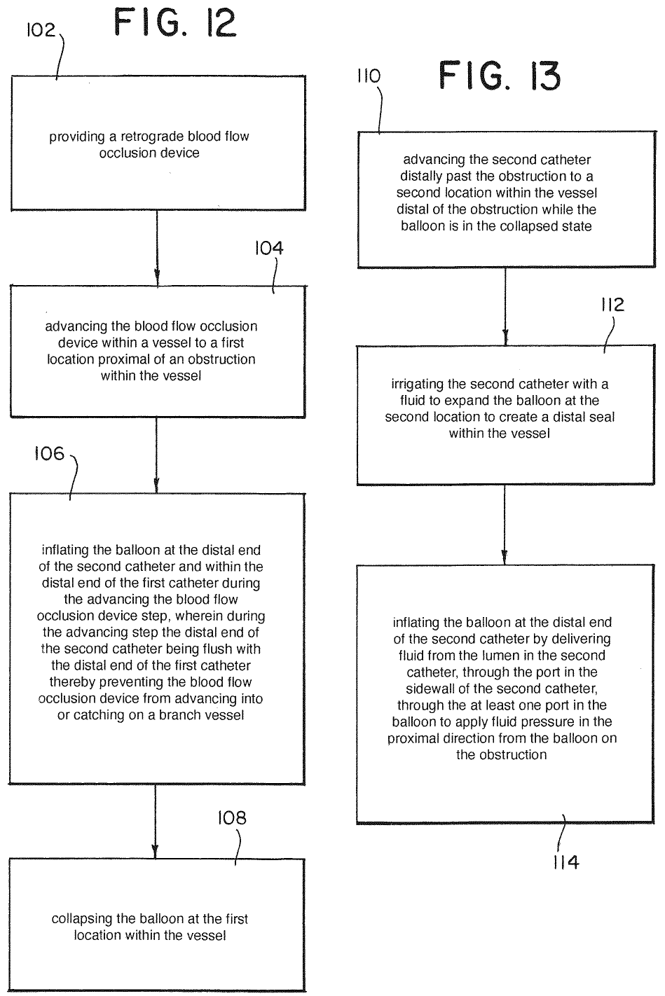

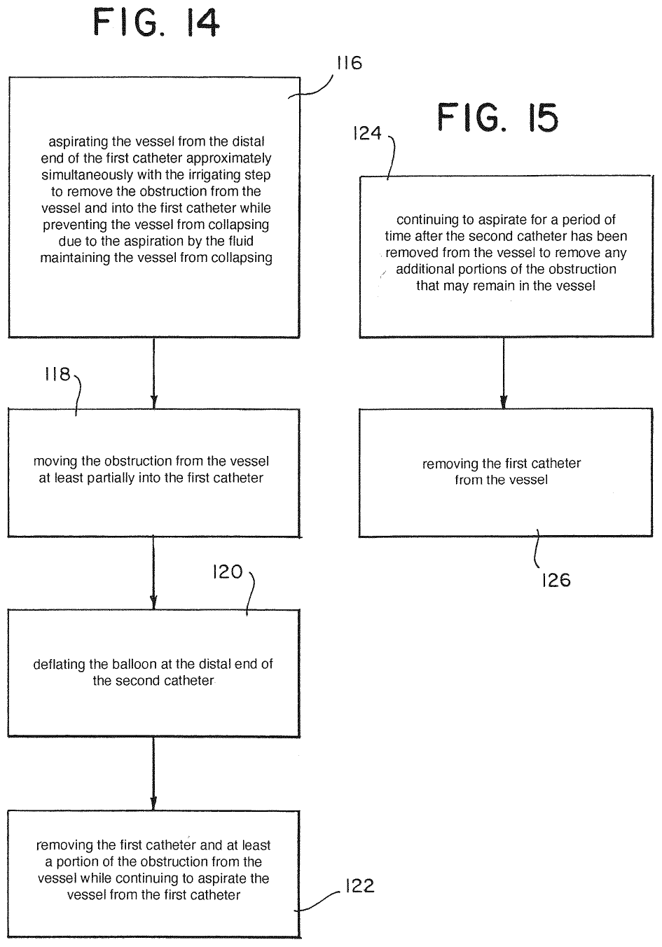

Referring now to FIGS. 12-15, a method of removing an obstruction from a vessel can include the following steps. Step 102 can be providing a retrograde blood flow occlusion device 10. Step 104 can include advancing the blood flow occlusion device 10 within a vessel to a first location 68 proximal of an obstruction 70 within the vessel. Step 106 can include inflating the balloon 28 at the distal end of the second catheter 20 and within the distal end 16 of the first catheter 12 during the advancing the blood flow occlusion device step, wherein during the advancing step the distal end 24 of the second catheter 20 being flush with the distal end 16 of the first catheter 12 thereby preventing the blood flow occlusion device 10 from advancing into or catching on a branch vessel 72. Step 108 can include collapsing the balloon 28 at the first location 68 within the vessel. Step 110 can include advancing the second catheter 20 distally past the obstruction 70 to a second location 74 within the vessel distal of the obstruction 70 while the balloon 28 is in the collapsed state. Step 112 can include irrigating the second catheter 20 with a fluid F to expand the balloon 28 at the second location 74 to create a distal seal within the vessel. Step 114 can include inflating the balloon 28 at the distal end 24 of the second catheter 20 by delivering fluid F from the lumen 26 in the second catheter 20, through the port 36 in the sidewall of the second catheter 20, through the at least one port 38 in the balloon 28 to apply fluid pressure in the proximal direction from the balloon 28 on the obstruction 70. Step 116 can include aspirating the vessel from the distal end 16 of the first catheter 12 approximately simultaneously with the irrigating step to remove the obstruction 70 from the vessel and into the first catheter 12 while preventing the vessel from collapsing due to the aspiration by the fluid F maintaining the vessel from collapsing.

The method can further include the following steps. Moving the obstruction 70 from the vessel at least partially into the first catheter 12. Deflating the balloon 28 at the distal end 24 of the second catheter 20. Removing the second catheter 20 and at least a portion of the obstruction 70 from the vessel while continuing to aspirate the vessel from the first catheter. Continuing to aspirate for a period of time after the second catheter has been removed from the vessel to remove any additional portions of the obstruction that may remain in the vessel. Removing the first catheter from the vessel.

Alternatively, the method can further include the following steps. Step 118 can include moving the obstruction 70 from the vessel at least partially into the first catheter 12. Step 120 can include deflating the balloon 28 at the distal end 24 of the second catheter 20. Step 122 can include removing the first catheter 12 at least a portion of the obstruction 70 from the vessel while continuing to aspirate the vessel from the first catheter. Step 124 can include continuing to aspirate for a period of time after the second catheter has been removed from the vessel to remove any additional portions of the obstruction that may remain in the vessel. Step 126 can include removing the first catheter from the vessel.

Aspects of the invention are also provided by the following numbered clauses:
Clause 1. A retrograde blood flow occlusion flushing device 10, comprising:
   a first catheter 12 having a proximal end 14, a distal end 16, and a lumen 18 extending through the first catheter 12 from the proximal end 14 to the distal end 16;
   a second catheter 20 disposed within the lumen 18 of the first catheter 12, the second catheter 20 having a proximal end 22, a distal end 24, and a lumen 26 extending through the second catheter 20 from the proximal end 22 to the distal end 24, the second catheter 20 being axially movable with respect to the first catheter 12, the second catheter 20 having an inflatable balloon 28 connected to an outer circumferential surface 30 of the second catheter 20 at the distal end 24 of the second catheter 20, the balloon 28 being movable between a collapsed state and an inflated state, the balloon 28 being fixedly connected to the outer circumferential surface 30 of the second catheter 20 at a proximal end 32 of the balloon and at a distal end 34 of the balloon, the second catheter 20 having a port 36 in the sidewall between the proximal end 32 of the balloon 28 and the distal end 34 of the balloon 28, the balloon 28 having at least one port 38 proximate the proximal end 32 of the balloon 28;
   wherein the balloon 28 is movable between the collapsed state and the inflated state by a fluid F deliverable through the lumen 18 of the second catheter 20 via the proximal end 22 and exiting the port 36 in the sidewall of the second catheter 20 to selectively move the balloon 28 between the collapsed state and the inflated state; in the inflated state, the fluid F being deliverable through the lumen 18 of the second catheter, through the port 36 in the sidewall of the second catheter 20 to inflate the balloon 28 and at least some of the fluid F exiting the inflated balloon 28 in the proximal direction through the at least one port 38 in the balloon 28.
Clause 2. The retrograde blood flow occlusion flushing device of clause 1, wherein the distal end 24 of the second catheter 20 is sealed such that the only openings in the second catheter are at the proximal end 22 and at the port 36 in the sidewall of the second catheter 20.
Clause 3. The retrograde blood flow occlusion flushing device of any of clauses 1-2, wherein the second catheter 20 has a first radiopaque marker 40 proximate to and proximal to the balloon 28.
Clause 4. The retrograde blood flow occlusion flushing device of any of clauses 1-3, wherein the second catheter 20 has a second radiopaque marker 42 proximate to and distal to the balloon 28.
Clause 5. The retrograde blood flow occlusion flushing device of any of clauses 1-4, wherein the second radiopaque marker 42 is disposed at the sealed distal end of the second catheter 20.
Clause 6. The retrograde blood flow occlusion flushing device of any of clauses 1-5, wherein the distal end 16 of the first catheter 12 has a radially outwardly flared opening 44.
Clause 7. A blood flow occlusion flushing device 10, comprising:
   a first catheter 12 having a proximal end 14, a distal end 16, and a lumen 18 extending through the first catheter 12 from the proximal end 14 to the distal end 16;
   a second catheter 20 disposed within the lumen 18 of the first catheter 12, the second catheter 20 having a proximal end 22, a distal end 24, and a lumen 26 extending through the second catheter 20 from the proximal end 22 to the distal end 24, the second catheter 20 being axially movable with respect to the first catheter 12, the second catheter 20 having a port 36 in the sidewall proximate the distal end 24 of the second catheter 20;
   a guidewire 46 disposed within the lumen 18 of the second catheter 20, the guidewire 46 being axially movable with respect to the second catheter 20, the guidewire having a proximal end 48 and a distal end 50, the guidewire 46 having a first diameter 52 that is less than an inner diameter 54 of the second catheter 20, the guidewire 46 having a radially outwardly projecting bump 56 proximate the distal end 50 of the guidewire 46, the radially outwardly projecting bump 56 having an outer diameter 58 that is approximately equal to an inner diameter 60 of the second catheter 20 at the distal end 24 of the second catheter 20 such that when the radially outwardly projecting bump 56 is aligned with the distal end 24 of the second catheter 20, the radially outwardly projecting bump 56 being in sealing contact with the inner diameter 60 of the second catheter 20 at the distal end 24 of the second catheter 20 such that a fluid F deliverable through the lumen 18 of the second catheter 20 via the proximal end 22 and exiting the port 36 in the sidewall of the second catheter 20.
Clause 8. The retrograde blood flow occlusion flushing device of clause 7, further comprising an inflatable balloon 28 connected to an outer circumferential surface 30 of the second catheter 20 at the distal end 24 of the second catheter 20, the balloon 28 being movable between a collapsed state and an inflated state, the inflatable balloon 28 being fixedly connected to the outer circumferential surface 30 of the second catheter 20 at a proximal end 32 of the balloon 28 and at a distal end 34 of the balloon 28.
Clause 9. The retrograde blood flow occlusion flushing device of any of clauses 1-8, wherein the balloon 28 having at least one port 38 between the proximal end 32 of the balloon and the distal end 34 of the balloon 28.
Clause 10. The retrograde blood flow occlusion flushing device of any of clauses 1-9, wherein the at least one port 38 in the balloon being disposed at the proximal end 32 of the balloon 28.
Clause 11. The retrograde blood flow occlusion flushing device of any of clauses 1-10, wherein the second catheter 20 has a first radiopaque marker 40 proximate to and proximal to the balloon 28.
Clause 12. The retrograde blood flow occlusion flushing device of any of clauses 1-11, wherein the second catheter 28 has a second radiopaque marker 42 proximate to and distal to the balloon 28.
Clause 13. The retrograde blood flow occlusion flushing device of any of clauses 1-12, wherein the distal end 16 of the first catheter has a radially outwardly flared opening 44.
Clause 14. The retrograde blood flow occlusion flushing device of any of clauses 1-13 wherein the radially outwardly projecting bump 56 is a radiopaque marker.
Clause 15. A blood flow occlusion flushing device 10, comprising:
   a first catheter 12 having a proximal end 14, a distal end 16, and a lumen 18 extending through the first catheter 12 from the proximal end 14 to the distal end 16;
   a second catheter 20 disposed within the lumen 18 of the first catheter 12, the second catheter 20 having a proximal end 22, a distal end 24, a first lumen 26 extending through the second catheter 20 from the proximal end 22 to the distal end 24, and a second lumen 62 extending through the second catheter 20 from the proximal end 22 to the distal end 24, the second catheter 20 being axially movable with respect to the first catheter 12, the second catheter 20 having a port 36 in the sidewall proximate the distal end 34 of the second catheter 20, the second catheter 20 having an inflatable balloon 28 connected to an outer circumferential surface 30 of the second catheter 20 at the distal end 24 of the second catheter 20, the balloon 28 being movable between a collapsed state and an inflated state, the balloon 28 being fixedly connected to the outer circumferential surface 30 of the second catheter 20 at a proximal end 32 of the balloon 28 and at a distal end 34 of the balloon 28, the second catheter 20 having a port 36 in the sidewall between the proximal end 32 of the balloon 28 and the distal end 34 of the balloon 28, the balloon 28 having at least one port 38 proximate the proximal end 32 of the balloon 28;
   a guidewire 46 disposed within the second lumen 62 of the second catheter 20, the guidewire 46 being axially movable with respect to the second catheter 20, the guidewire 20 having a proximal end 64 and a distal end 66; and
   wherein the balloon 28 is movable between the collapsed state and the inflated state by a fluid F deliverable through the lumen 18 of the second catheter 20 via the proximal end 22 and exiting the port 36 in the sidewall of the second catheter 20 to selectively move the balloon 28 between the collapsed state and the inflated state; in the inflated state, the fluid F being deliverable through the first lumen 18 of the second catheter, through the port 36 in the sidewall of the second catheter 20 to inflate the balloon 28 and at least some of the fluid F exiting the inflated balloon 28 in the proximal direction through the at least one port 38 in the balloon 28.
Clause 16. The retrograde blood flow occlusion flushing device of clause 15, wherein the first lumen 26 and the second lumen 62 in the second catheter 20 are co-axial.
Clause 17. The retrograde blood flow occlusion flushing device of any of clauses 15-16, wherein the second lumen 62 in the second catheter 20 is offset with respect to the first lumen 26 in the second catheter 20.
Clause 18. The retrograde blood flow occlusion flushing device of any of clauses 15-17, wherein the distal end 24 of the second catheter 20 is sealed such that the only openings in the second catheter 20 are at the proximal end 22 and at the port 36 in the sidewall of the second catheter 20.
Clause 19. The retrograde blood flow occlusion flushing device of any of clauses 15-18, wherein the second catheter 20 has a first radiopaque marker 40 proximate to and proximal to the balloon 28.
Clause 20. The retrograde blood flow occlusion flushing device of any of clauses 15-19, wherein the second catheter 20 has a second radiopaque marker 42 proximate to and distal to the balloon 28.
Clause 21. The retrograde blood flow occlusion flushing device of any of clauses 15-20, wherein the second radiopaque marker 42 is disposed at the sealed distal end 24 of the balloon.
Clause 22. The retrograde blood flow occlusion flushing device of any of clauses 15-21, wherein the distal end 16 of the first catheter has a radially outwardly flared opening 44.
Clause 23. A method of removing an obstruction from a vessel, the method comprising the steps of:
   providing a retrograde blood flow occlusion device 10 comprising:
   a first catheter 12 having a proximal end 14, a distal end 16, and a lumen 18 extending through the first catheter from the proximal end to the distal end;
   a second catheter 20 disposed within the lumen 18 of the first catheter 12, the second catheter 20 having a proximal end 22, a distal end 24, and a lumen 26 extending through the second catheter 20 from the proximal end to the distal end, the second catheter 20 being axially movable with respect to the first catheter 12, the second catheter 20 having an inflatable balloon 28 connected to an outer circumferential surface 30 of the second catheter 20 at the distal end 24 of the second catheter 20, the balloon 28 being movable between a collapsed state and an inflated state, the balloon 28 being fixedly connected to the outer circumferential surface 30 of the second catheter 20 at a proximal end 32 of the balloon 28 and at a distal end 34 of the balloon 28, the second catheter 20 having a port 36 in the sidewall between the proximal end 32 of the balloon 28 and the distal end 34 of the balloon 28, the balloon 28 having at least one port 38 proximate the proximal end 32 of the balloon 28;
   advancing the blood flow occlusion device 10 within a vessel to a first location 68 proximal of an obstruction 70 within the vessel;
   inflating the balloon 28 at the distal end of the second catheter 20 and within the distal end 16 of the first catheter 12 during the advancing the blood flow occlusion device step, wherein during the advancing step the distal end 24 of the second catheter 20 being flush with the distal end 16 of the first catheter 12 thereby preventing the blood flow occlusion device 10 from advancing into or catching on a branch vessel 72;
   collapsing the balloon 28 at the first location 68 within the vessel;
   advancing the second catheter 20 distally past the obstruction 70 to a second location 74 within the vessel distal of the obstruction 70 while the balloon 28 is in the collapsed state;
   irrigating the second catheter 20 with a fluid F to expand the balloon 28 at the second location 74 to create a distal seal within the vessel;
   inflating the balloon 28 at the distal end 24 of the second catheter 20 by delivering fluid F from the lumen 26 in the second catheter 20, through the port 36 in the sidewall of the second catheter 20, through the at least one port 38 in the balloon 28 to apply fluid pressure in the proximal direction from the balloon 28 on the obstruction 70; and
   aspirating the vessel from the distal end 16 of the first catheter 12 approximately simultaneously with the irrigating step to remove at least a portion of the obstruction 70 from the vessel and into the first catheter 12 while preventing the vessel from collapsing due to the aspiration by the fluid F maintaining the vessel from collapsing.
Clause 24. The method of removing an obstruction from a vessel according to clause 23, further comprising the step of:
   moving the obstruction 70 from the vessel at least partially into the first catheter 12;
   deflating the balloon 28 at the distal end 24 of the second catheter 20;
   removing the second catheter 20 and at least a portion of the obstruction 70 from the vessel while continuing to aspirate the vessel from the first catheter;
   continuing to aspirate for a period of time after the second catheter has been removed from the vessel to remove any additional portions of the obstruction that may remain in the vessel; and
   removing the first catheter from the vessel.
Clause 25. A retrograde blood flow occlusion flushing device 10, comprising:
   a first catheter 20 having a proximal end 22, a distal end 24, and a lumen 26 extending through the first catheter 20 from the proximal end 22 to the distal end 24, the first catheter 20 having an inflatable balloon 28 connected to an outer circumferential surface 30 of the first catheter 20 at the distal end 24 of the first catheter 20, the balloon 28 being movable between a collapsed state and an inflated state, the balloon 28 being fixedly connected to the outer circumferential surface 30 of the first catheter 20 at a proximal end 32 of the balloon and at a distal end 34 of the balloon, the first catheter 20 having a port 36 in the sidewall between the proximal end 32 of the balloon 28 and the distal end 34 of the balloon 28, the balloon 28 having at least one port 38 proximate the proximal end 32 of the balloon 28;
   wherein the balloon 28 is movable between the collapsed state and the inflated state by a fluid F deliverable through the lumen 18 of the first catheter 20 via the proximal end 22 and exiting the port 36 in the sidewall of the first catheter 20 to selectively move the balloon 28 between the collapsed state and the inflated state; in the inflated state, the fluid F being deliverable through the lumen 18 of the first catheter, through the port 36 in the sidewall of the first catheter 20 to inflate the balloon 28 and at least some of the fluid F exiting the inflated balloon 28 in the proximal direction through the at least one port 38 in the balloon 28.
Clause 26. The retrograde blood flow occlusion flushing device of clause 25, wherein the distal end 24 of the first catheter 20 is sealed such that the only openings in the first catheter are at the proximal end 22 and at the port 36 in the sidewall of the first catheter 20.
Clause 27. The retrograde blood flow occlusion flushing device of any of clauses 25-26, wherein the first catheter 20 has a first radiopaque marker 40 proximate to and proximal to the balloon 28.
Clause 28. The retrograde blood flow occlusion flushing device of any of clauses 25-27, wherein the first catheter 20 has a second radiopaque marker 42 proximate to and distal to the balloon 28.
Clause 29. The retrograde blood flow occlusion flushing device of any of clauses 25-28, wherein the second radiopaque marker 42 is disposed at the sealed distal end of the first catheter 20.

The descriptions contained herein are examples of embodiments of the disclosure and are not intended in any way to limit the scope of the disclosure. As described herein, the disclosure contemplates many variations and modifications, and those variations and modifications will be apparent to those having skilled in the pertinent art according to the teachings of this disclosure are intended to be within the scope of the claims which follow.

What is claimed is:

1. A retrograde blood flow occlusion flushing device, comprising:
a first catheter having a proximal end, a distal end, and a lumen extending through the first catheter from the proximal end to the distal end;
a second catheter disposed within the lumen of the first catheter, the second catheter having a proximal end, a distal end, and a lumen extending through the second catheter from the proximal end to the distal end, the second catheter being axially movable with respect to the first catheter, the second catheter having an inflatable balloon connected to an outer circumferential surface of the second catheter at the distal end of the second catheter, the balloon being movable between a collapsed state and an inflated state, the balloon being fixedly connected to the outer circumferential surface of the second catheter at a proximal end of the balloon and at a distal end of the balloon, the second catheter having a port in the sidewall between the proximal end of the balloon and the distal end of the balloon, the balloon having at least one port proximate the proximal end of the balloon;
wherein the balloon is movable between the collapsed state and the inflated state by a fluid deliverable through the lumen of the second catheter via the proximal end and exiting the port in the sidewall of the second catheter to selectively move the balloon between the collapsed state and the inflated state; in the inflated state, the fluid being deliverable through the lumen of the second catheter, through the port in the sidewall of the second catheter to inflate the balloon and at least some of the fluid exiting the inflated balloon in the proximal direction through the at least one port in the balloon.

2. The retrograde blood flow occlusion flushing device of claim 1, wherein the distal end of the second catheter is sealed such that the only openings in the second catheter are at the proximal end and at the port in the sidewall of the second catheter.

3. The retrograde blood flow occlusion flushing device of claim 2, wherein the second catheter has a first radiopaque marker proximate to and proximal to the balloon.

4. The retrograde blood flow occlusion flushing device of claim 3, wherein the second catheter 20 has a second radiopaque marker proximate to and distal to the balloon.

5. The retrograde blood flow occlusion flushing device of claim 4, wherein the second radiopaque marker is disposed at the sealed distal end of the second catheter.

6. The retrograde blood flow occlusion flushing device of claim 1, wherein the distal end of the first catheter has a radially outwardly flared opening.

7. A blood flow occlusion flushing device, comprising:
a first catheter having a proximal end, a distal end, and a lumen extending through the first catheter from the proximal end to the distal end;
a second catheter disposed within the lumen of the first catheter, the second catheter having a proximal end, a distal end, and a lumen extending through the second catheter from the proximal end to the distal end, the second catheter being axially movable with respect to the first catheter, the second catheter having a port in the sidewall proximate the distal end of the second catheter;
a guidewire disposed within the lumen of the second catheter, the guidewire being axially movable with respect to the second catheter, the guidewire having a proximal end and a distal end, the guidewire having a first diameter that is less than an inner diameter of the second catheter, the guidewire having a radially outwardly projecting bump proximate the distal end of the guidewire, the radially outwardly projecting bump having an outer diameter that is approximately equal to an inner diameter of the second catheter at the distal end of the second catheter such that when the radially outwardly projecting bump is aligned with the distal end of the second catheter, the radially outwardly projecting bump being in sealing contact with the inner diameter of the second catheter at the distal end of the second catheter such that a fluid deliverable through the lumen of the second catheter via the proximal end and exiting the port in the sidewall of the second catheter.

8. The retrograde blood flow occlusion flushing device of claim 7, further comprising an inflatable balloon connected to an outer circumferential surface of the second catheter at the distal end of the second catheter, the balloon being movable between a collapsed state and an inflated state, the inflatable balloon being fixedly connected to the outer circumferential surface of the second catheter at a proximal end of the balloon and at a distal end of the balloon.

9. The retrograde blood flow occlusion flushing device of claim 8, wherein the balloon having at least one port between the proximal end of the balloon and the distal end of the balloon.

10. The retrograde blood flow occlusion flushing device of claim 9, wherein the at least one port 38 in the balloon being disposed at the proximal end of the balloon.

11. The retrograde blood flow occlusion flushing device of claim 10, wherein the second catheter has a first radiopaque marker proximate to and proximal to the balloon.

12. The retrograde blood flow occlusion flushing device of claim 11, wherein the second catheter has a second radiopaque marker proximate to and distal to the balloon.

13. The retrograde blood flow occlusion flushing device of claim 7, wherein the distal end of the first catheter has a radially outwardly flared opening.

14. The retrograde blood flow occlusion flushing device of claim 7, wherein the radially outwardly projecting bump is a radiopaque marker.

15. A retrograde blood flow occlusion flushing device, comprising:
a first catheter having a proximal end, a distal end, and a lumen extending through the first catheter from the proximal end to the distal end, the first catheter having an inflatable balloon connected to an outer circumferential surface of the first catheter at the distal end of the first catheter, the balloon being movable between a collapsed state and an inflated state, the balloon being fixedly connected to the outer circumferential surface of the first catheter at a proximal endof the balloon and at a distal end 34 of the balloon, the first catheter 20 having a port 36 in the sidewall between the proximal end of the balloon and the distal end of the balloon, the balloon having at least one port proximate the proximal end of the balloon;
wherein the balloon is movable between the collapsed state and the inflated state by a fluid deliverable through the lumen of the first catheter via the proximal end and exiting the port in the sidewall of the first catheter to selectively move the balloon between the collapsed state and the inflated state; in the inflated state, the fluid being deliverable through the lumen of the first catheter, through the port in the sidewall of the first catheter to inflate the balloon and at least some of the fluid exiting the inflated balloon in the proximal direction through the at least one port in the balloon.

16. The retrograde blood flow occlusion flushing device of claim 15, wherein the distal end of the first catheter is sealed such that the only openings in the first catheter are at the proximal end and at the port in the sidewall of the first catheter.

17. The retrograde blood flow occlusion flushing device of claim 16, wherein the first catheter has a first radiopaque marker proximate to and proximal to the balloon.

18. The retrograde blood flow occlusion flushing device of claim 17, wherein the first catheter has a second radiopaque marker proximate to and distal to the balloon.

19. The retrograde blood flow occlusion flushing device of claim 18, wherein the second radiopaque marker is disposed at the sealed distal end of the first catheter.
